(19) United States
(12) Patent Application Publication
(10) Pub. No.: US 2003/0004467 A1
(43) Pub. Date: Jan. 2, 2003

(54) Multi-dose syringe driver

(75) Inventors: James R. Musick, Conifer, CO (US); Jon P. Page, Broomfield, CO (US)

Correspondence Address: Gary J. Connell, SHERIDAN ROSS P.C., 1560 Broadway, Suite 1200, Denver, CO, 80202-5141, US

(73) Assignee: Vitro Diagnostics, Inc.

(21) Appl. No.: 10/175,501

(22) Filed: Jun. 18, 2002

Related U.S. Application Data
(63) Continuation of application No. 09/660,593, filed on Sep. 13, 2000, now abandoned.
(60) Provisional application No. 60/153,756, filed on Sep. 13, 1999.

(51) Int. Cl. A61M 5/00

(52) U.S. Cl. 604/218

(57) ABSTRACT

Disclosed is a driver for use with administering multiple doses of a compound contained in a cartridge. The driver has a base, a position selector, and a plunger. The plunger shaft includes projections which engage the position selector so the plunger advances to provide a single dose of the compound. The plunger shaft is rotated to align the plunger projections with the position selector to administer a subsequent dose of the compound.

### CROSS-REFERENCE TO RELATED APPLICATIONS

This application claims priority of U.S. Provisional Patent Application Serial No. 60/153,756, filed Sep. 13, 1999, which is incorporated herein by reference in its entirety.

### FIELD OF THE INVENTION

The invention is a multi-dosing syringe allowing sequential injection of identical or non-identical volumes of liquids or dissolvable solids & liquids contained with the syringe. More particularly, the invention allows for multi-dose injection of liquid or combined liquid/solid products through the use of a driver that uses a novel mechanism to deliver multiple injectable doses.

### BACKGROUND OF THE INVENTION

Several different devices are available which allow for multiple injection of the same or different doses from a syringe or other component containing liquid pharmaceutical products. Some devices utilize a standard disposable syringe mounted in a frame designed and driven by an electrical/mechanical pump to infuse multiple volumes of liquid pharmaceutical. These devices allow for electronic control of flow rate, which is usually relatively low for infusion applications. Multiple dosing is achieved through electronic pump control resulting in a desired syringe plunger displacement, at a specified rate and at a specified time. While suited to infusion in a hospital setting, multiple dose drug delivery through use of an infusion pump is costly, and limited by power and maintenance requirements.

U.S. Pat. No. 5,722,956 and International Patent Application WO 97/07838 provide an invention allowing multi-dose administration through use of a spring-driven mechanical device that attaches to a standard disposable syringe. This device also provides a length of micro-bore tubing through which fluid flows after leaving the syringe to provide resistance to limit flow. This device contains rails and stops allowing insertion of a syringe filled with liquid between its plunger and the base of the syringe. In this loaded position, the spring driver device is located at the proximal end of the syringe plunger. The rail at the plunger end is then manually compressed towards the base of syringe which advances the driver to a detent position on the track allowing insertion of stopping pawl which is driven laterally by another smaller spring. This action results in compression of the driver spring axially toward the syringe plunger, hence applying force to the plunger through an internal piston. Fluid will be expelled from the syringe needle cannula at a rate dependent on the spring force and the flow resistance of the cannula. The detents are positioned to prevent initiation of a subsequent dose until the prior one has been expelled from the syringe. The next dose is initiated by further manual compression of the driver tracks between the syringe base and plunger. The device usually requires two hands to initiate injection of a drug dose, is particularly suited to infusion of a drug in a hospital setting, is restricted to a particular size of syringe, e.g., 60 mL and has several working metallic parts.

Procedures for the production of reusable automatic injectors have been described involving spring driven or manual injectors with retractable needle shields (U.S. Pat. Nos. 4,664,653, 5,540,664 and 5,665,071). These devices are typically used for emergency injection of epinephrine in the treatment of allergic reactions and other life-threatening conditions. Since such applications often require more than one epinephrine treatment, these devices are reusable simply by the removal of the spent drug-containing component of the device and its replacement with another drug-filled device. However, while these devices allow multi-dose injection, they require replacement of the drug-containing component of the device for each dose.

U.S. Pat. No. 5,232,459 provides methods to make a multi-dose syringe comprising an internal cartridge containing the liquid injectable product and a outer sleeve containing reciprocal plunger stop positions which allow for multi-dosing. The position of the plunger stopping surfaces may be changed by rotation of a threaded coupling which changes the relative positions of the stopping surfaces, the distance the plunger is displaced and hence the volume injected. A unidirectional click-stop mechanism allows rotation to the next detent position. The syringe plunger may then be depressed to effect the next injection of drug which is the same volume as the first. Subsequent rotation of the plunger likewise allows injection of subsequent doses of the drug.

Dose-indicating pens allow multi-dose injections of different doses of liquid pharmaceutical product (International Patent Number 05226896). These devices contain a dose-indicating window showing the dose injected. Some are coupled to a calibrated piston that determines the displacement of the syringe plunger upon injection from the syringe and hence the amount of medication injected.

Several pharmaceutical agents are relatively unstable in their liquid form, especially those of a biological nature including hormones, proteins and other agents. One solution to the problem of unstable pharmaceutical agents has been to utilize dual-chambered glass cartridges. One chamber contains the active pharmaceutical agent in a freeze-dried form, while the other chamber contains a liquid diluent such as physiological saline. In this form, the pharmaceutical product is usually quite stable and has a long shelf-life in a variety of environmental conditions. Initiation of use results in intermixing between the two chambers, either through penetration of the intervening septum with a needle spike (U.S. Pat. No. 5,281,198) or by displacement of the septum into a region of the glass cartridge barrel containing an axially oriented bypass channel that is longer than the width of the septum (European Patent Numbers 0718002 and 0440846). The device of U.S. Pat. No. 5,281,198 further extends the dual chamber glass syringe to include a driver device for multi-dose injections. The multi-dosing mechanism involves a ratchet plunger that moves a distance determined by a dosing key stop along the length of the ratchet plunger. The dosing key stop may be positioned, preferably by a pharmacist, to deliver different volumes. Multiple injections occur by ejection of the same volume. However, this device is complex, containing over 140 components, which precludes its use as a multi-dose, disposable injection system.

Therefore, a need exists for a disposable multi-dose syringe having a simple driver made of few parts that can be easily produced, for example, by use of plastic injection molding procedures. The present invention satisfies this need and provides related advantages as well.

### SUMMARY OF THE INVENTION

One embodiment of the present invention is a driver for delivering multiple doses of a compound from a cartridge. The driver includes a threaded base which is adapted for attachment to a cartridge. The driver also includes a position selector. The position selector includes external threads for seating with the threads of the base, a central opening extending through the position selector, and a keyway in the central opening. Finally, the driver includes a plunger which has a shaft, a distal end, and a proximal end. The plunger extends through the central opening of the position selector and is longer than the position selector. The plunger also includes projections for passage through the keyway on the position selector.

In alternative aspects of this embodiment, the projections on the plunger shaft can be separated from each other axially and at different angles to the axis of the plunger shaft. Also, the projections can either be spaced equally along a longitudinal axis of the plunger shaft or can spaced along the longitudinal axis of the plunger shaft in a ratio corresponding to desired dosages of the composition being delivered. The position selector can also include a window to allow visual access to position indicators on the plunger shaft. The projections can include a proximal end and a distal end wherein the proximal end of the projection has a greater radial profile than the distal end. In this manner, insertion of the distal end of the projection into the keyway can produce an audible or tactile stimulus.

A further embodiment of the present invention is a multiple dose administration device which includes the driver described above and a cartridge. The cartridge can either include a single chamber or a dual chamber having a proximal chamber containing a liquid agent and a distal chamber containing a dried agent. For example, compounds that can be delivered by such an administration device can be selected from FSH, insulin, growth hormone, interferon, colony stimulating factor, arthropoitene, steroid hormones, and allergens.

A further embodiment of the invention si a method for ejecting a compound from a multiple dose syringe which includes attaching a cartridge containing the compound to the driver described above. The position selector is rotated to align a first projection with the keyway of the position selector. The plunger is then depressed until a second projection impacts the position selector, thereby ejecting a portion of the compound. Further the position selector is rotated to align the second projection of the plunger shaft with the keyway of the position selector. The plunger is then depressed until a third projection impacts the position selector to eject another portion of the compound.

In this method, the cartridge can include a first and second chamber having a diluent and a dried compound, respectively. This embodiment of the method further includes advancing the position selector into the threaded base to reconstitute the dried compound with the diluent. The method can also include the step of attaching a needle to a distal end of the cartridge and further advancing the position selector to purge air from the cartridge. Finally, the method can include inserting the needle into a patient.

A further embodiment of the present invention includes a driver for delivering multiple doses of a compound from a cartridge. The driver includes a base adapted for attachment to a cartridge. The base includes a central opening extending through the base and a keyway in the central opening. The driver further includes a plunger which extends through the central opening of the base and is longer than the base. The plunger includes projections for passage through the keyway.

### BRIEF DESCRIPTION OF DRAWINGS

FIG. 1A is a cross-sectional view of the driver of the present invention attached to a dual-chambered cartridge ready for use;

FIG. 1B is an end view of the position selector of the present invention of FIG. 1A as viewed from section “A-A” of FIG. 1A;

FIG. 2A is a side view of the driver of the present invention shown in FIG. 1;

FIG. 2B is an proximal end view of present invention of FIG. 2A;

FIG. 3 is a cross-sectional view of the driver of the present invention attached to a dual-chamber cartridge during reconstitution;

FIG. 4 is a cross-sectional view of the driver of the present invention and attached dual-chambered cartridge at the end of reconstitution;

FIG. 5 is a cross-sectional view of the driver of the present invention and attached dual-chambered cartridge at the end of the air purge; and

FIG. 6 is a cross-sectional view of the driver of the present invention and the attached dual-chambered cartridge at the end of the first delivery.

### DETAILED DESCRIPTION OF THE INVENTION

The device of the present invention is a simple driver device composed of relatively few parts that can be produced through use of plastic injection molding procedures. The driver uses a novel mechanism to generate multiple injection doses. The present device is further distinct from known devices by the mechanism through which injection volumes are determined during multi-dosing operations. For example, while the device of U.S. Pat. No. 5,232,459 uses rotation of a threaded coupling which changes the relative positions of the stopping surfaces, in the present device, the volume of each injection is pre-determined by the axial positioning of reciprocal stops located along the shaft of syringe plunger. The driver is manufactured to allow its direct attachment to a cartridge, most preferably a dual-chambered cartridge wherein the desired agent is stored in a dry form and separate from a liquid diluent. Prior to engagement of the multi-dose delivery feature of the device, syringe plunger movements are utilized to fully reconstitute the dried agent.

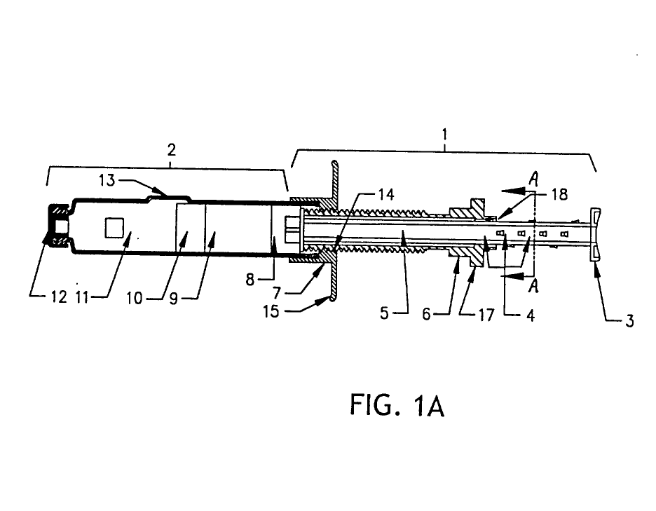

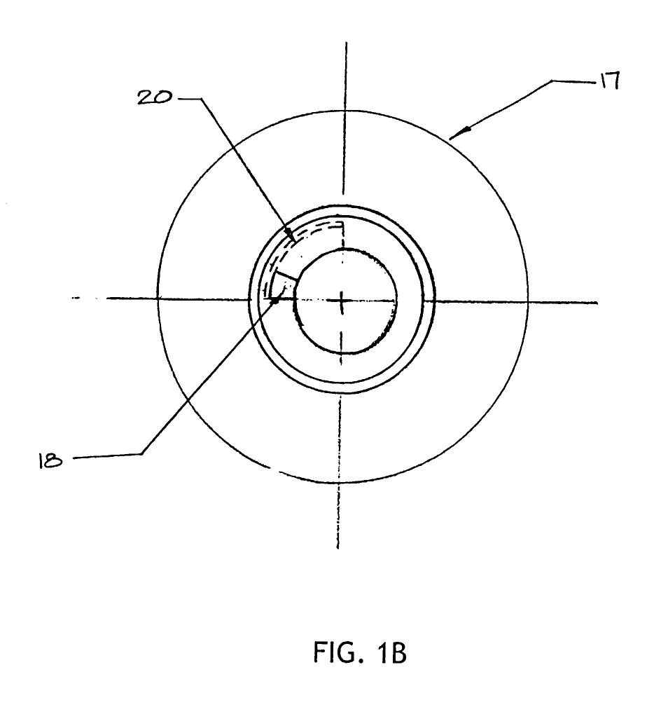

The multi-dose syringe of the present invention is shown in FIGS. 1-7. It consists of a female threaded base that attaches to the proximal end of a pre-filled cartridge, e.g. a dual-chambered cartridge. The base contains an oblong extension plate similar to that of a standard syringe. This is used to support the index and second finger when thumb pressure is applied to the plunger. An externally threaded position selector, which serves as a sleeve for the plunger, is screwed into the base. A knurled screw knob containing a dose indicating window is attached to the proximal end of the position selector through an ultrasonic weld joint. The plunger, containing laterally projecting plunger reciprocal stops on its exposed surface, fits tightly within the sleeve, extends into the glass cartridge and is attached to a proximal septum, tightly fit within the cartridge. The driver device as illustrated in FIGS. 1 to 7 is attached to a wet/dry, dual-chambered cartridge, while other embodiments of the present invention involve attachment to other types of cartridges.

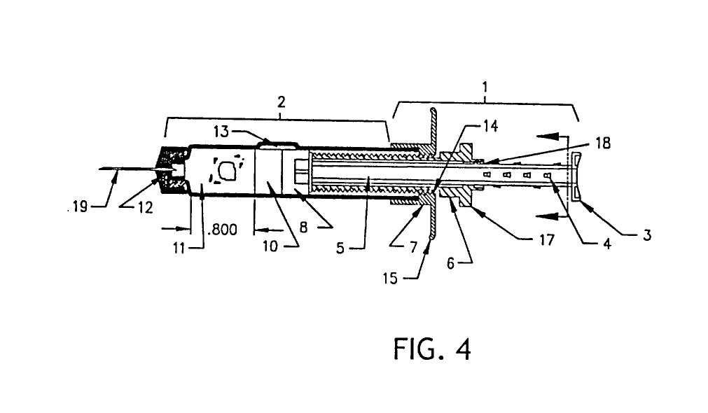

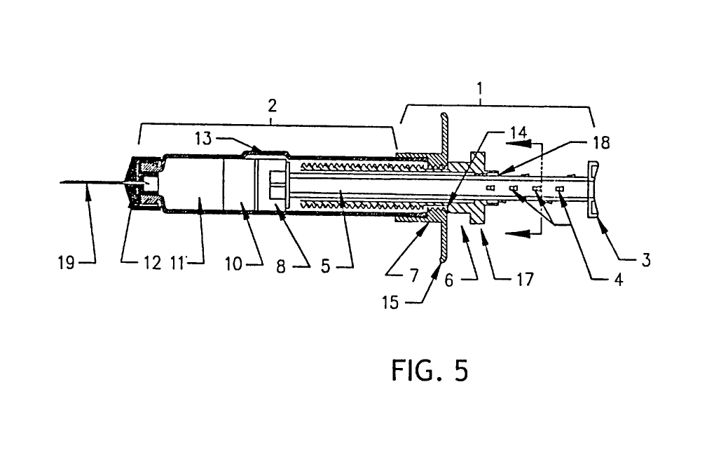

The driver of the present invention allows two distinct mechanisms for proximal to distal movement of the syringe plunger: screw-driven plunger displacement, and step-wise plunger displacement. Screw-driven plunger displacement is activated by clockwise rotation of the position selector as shown in FIG. 4. During use of the device, a needle is first attached to the distal needle fitting of the cartridge. Plunger movements activated by clockwise rotation of the plunger sleeve are most preferably used for reconstitution of the drug contained within a wet/dry cartridge. Distal displacement of the distal septum to the region of the cartridge bypass channel allows fluid to move from the diluent chamber past the proximal septum into the agent-containing chamber containing the dried agent (FIG. 4). Further distal movement of the plunger completes the reconstitution of the agent as the head of the threaded position selector seats into the base. At this position, the threads of the position selector disengage the corresponding female threads of the base as shown in FIG. 5.

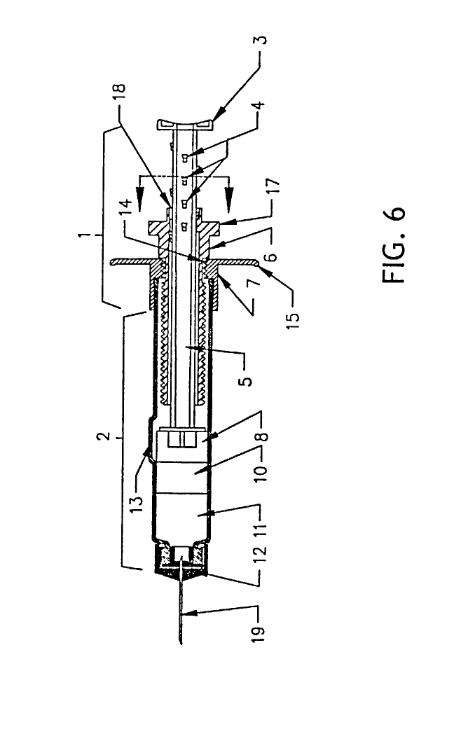

Further distal displacement of the plunger is step-wise, activated by, for example, thumb force applied by hand, between the base and the proximal end of the plunger. Each step results in incremental displacement of the syringe plunger. The first step is used to purge residual air from the vertically oriented syringe followed by sequential ejections through a needle attached to distal needle fitting of the cartridge, which is the multi-dose injection feature of the device. The mechanism of step-wise displacement is characterized by reciprocal stops comprised of rod-like, axially oriented protrusions on the plunger shaft. The reciprocal stops are positioned at a fixed angle relative to the plunger axis, e.g., 90°. When properly aligned, as indicated by the display within the dose-indicating window, these stops are designed to fit into a notch, also referred to as a keyway on the proximal surface of the position selector, allowing unidirectional distal movement of the plunger by a fixed increment, the increment determined by the axial distance separating two reciprocating stops, for example, 0.126 inches in FIG. 1. A dose-indicating window on the proximal end of the position selector shows which dose of the sequence has been injected. This window also has a position to indicate that the air purge has been completed (FIG. 6). The most distal reciprocal stop enters an internal keyway within the position selector, while the proximal reciprocal stop impacts the proximal surface of the position selector itself to limit displacement of the plunger (See FIG. 7).

The keyway, or notch, is an area of extended radius of the central aperture of the position selector through which the plunger stops move. The radial dimension of the extended radius is typically slightly larger than the radial dimension of the corresponding position stops to allow passage of the projecting stops through the keyway. If the projection stops are formed such that the proximal end of the stop has a greater radial profile than the distal end of the stop, the plunger may be prevented from moving in the proximal direction once the stop is properly received into the keyway. In this embodiment, the keyway radius is slightly less than the radius of the proximal end of the stop. As shown in FIG. 1A, the distal surface of the keyway is provided with a step 20 such that a portion of the internal diameter of the position selector below the keyway is slightly greater than the radius of the proximal end of the stop. The interaction of the undercut surface of the step 20 and the proximal end of the stop provides the user with a audible or tactile signal that the plunger has been advanced the proper distance.

An additional projection can also be provided distally of the step 20 such that the plunger is prevented from moving in the proximal and distal directions while the plunger is rotated. In this embodiment another opening, similar to the keyway, is provided in this lower projection to allow the plunger to be advanced in the distal direction once the plunger has been properly rotated.

The present invention provides several benefits. First, the driver device consists of only three parts. For pharmaceutical applications, the driver is particularly suited for construction from sterilizable amorphous medical plastic material as is used in injection molding of medical devices. Production by injection molding allows for cost-effective manufacture at large scales. Hence, the device may be disposed after use. The driver has multiple applications since it may be designed for attachment to a variety of cartridges, plastic or glass that contain pharmaceutical or non-pharmaceutical agents in a variety of formulations. Given that an injectable pharmaceutical agent is stable as a liquid, the driver offers multi-dosing capability through the step-wise advancement mechanism of plunger displacement, together with screw-driven plunger displacement to purge air from the drug compartment. Most preferably, the driver is suited for injection of biotherapeutic products stored in dual-chambered cartridge containers, wherein the labile biological material is maintained in a freeze-dried or otherwise stabilized formulation while the fluid used for reconstitution is stored in the other chamber. Since the device can be operated with a single hand and highly purified biological pharmaceuticals may be administered through subcutaneous injection, the device is applicable to self-administration of biotherapeutic agents. There are several drugs or biological compounds suitable for storage and delivery from such a dual-chambered device including, but not limited to, FSH, insulin, growth hormone, interferon, colony stimulating factor, erythropoietin, steroid hormones such as prednilisone and an allergen or mixture thereof. The present device is ideally applied to those drug substances which must be taken daily for extended periods of time. Also, there are many applications for such a device in the delivery of non-pharmaceutical substances for use in industrial applications, crafts and other non-pharmaceutical uses readily determined by those skilled in the art.

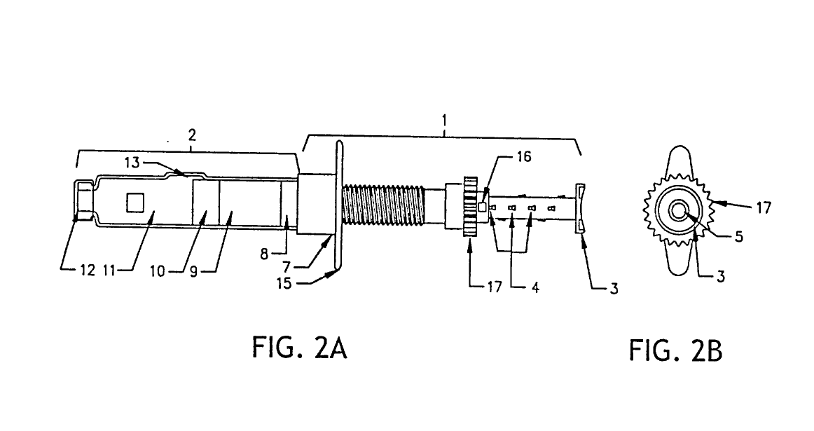

The novel mechanism of the present syringe provides for continuous screw-driven displacement of the syringe plunger (3) followed by step-wise displacement for injection of multiple doses of the same or different volumes. FIG. 1 shows the components of the multi-dose driver. The base (7) is shown attached to a dual-chambered cartridge (2). The base (7) contains an internal female thread (14) and an oblong plate extension (15) (FIG. 2) which is used to apply thumb pressure to the plunger (3). The threaded position selector (6) is externally threaded with a pitch identical to that of the base (7). The position selector (6) also serves as a sleeve housing for the plunger (3) which extends through the position selector (6) and is attached to the proximal septum (8) of the dual-chambered cartridge (2) through a cross fitting. The position selector (6) has a dose-indicating window (16) and a knurled screw knob (17) to facilitate its clockwise rotation (FIGS. 1 and 2).

For pharmaceutical applications, the device is designed for construction from sterilizable amorphous medical plastic material, as is used in injection molding of medical devices including, but not limited to, polycarbonate, ABS, Delrin®, and nylon. The materials used in the construction of the device are of suitable grade for the intended application, including USP grades for pharmaceutical applications or lessor grades for industrial or other non-pharmaceutical applications. The device may be used in such non-pharmaceutical applications requiring the ejection from a cartridge of such materials as epoxy, lubricants, glues, or cake frosting.

The use of the novel syringe of the present invention involves several functional operations including (a) product storage, (b) reconstitution of dried agent by diluent addition within a dual-chambered cartridge, (c) purging of residual air, and (d) multi-dose injection. Each of these functions of the present invention are described in detail below.

A. Product Storage

The multi-dose syringe driver is attachable to a cylindrical cartridge containing a desired agent. The section of the threaded base of the driver that receives the cartridge is adapted for attachment to the cartridge. The attachment may be accomplished by a friction fit, which may include a natural or synthetic gasket, threaded attachment, a snap ring to accommodate a corresponding groove in the cartridge, or the like. The cartridge can be single-chambered containing an injectable formulation or, preferably, dual-chambered in which the diluent chamber (9) contains a liquid diluent while the agent-containing chamber (11) contains a dried formulation, as shown in FIG. 1. The distal end of this cartridge is sealed by a distal needle fitting (12) made of a rubber seal and also accommodates the attachment of a needle (19) for injection. Displacement of the distal septum (10) into a region of the cartridge barrel (2) containing an axially oriented bypass channel (13) that is longer than the width of the distal septum (10) results in fluid flow past the bypass channel (13) and reconstitution of dried product contained within the agent-containing chamber (11).

This embodiment may also be applied to non-pharmaceutical agents, stored in dried and/or liquid form. Alternatively, the cartridge attached to the driver may contain any aqueous liquid agent. Furthermore, the multi-dose driver may be attached to cartridges containing viscous agents, including for example, adhesives and other agents with industrial applications. A limitation of this later embodiment is limited flow due to high agent viscosity and/or resistance to flow at the point of ejection. However, those skilled in the art can readily determine the upper range of viscosity and/or flow resistance useful in the present device.

The driver device attached to any cylinder containing any agent may be treated by methods well-known in the art to preserve the device and its agents for storage, shipment and warehousing. For instance, pharmaceutical applications would require that all components and the filling process be performed aseptically to eliminate bacterial contamination. Alternatively, some components such as the driver device of the present invention may be assembled in a non-sterile environment and then sterilized by known processes, including, for example, exposure to gamma rays or ethylene trioxide. A finished device may be sealed by methods known to those skilled in the art to preserve sterility or other attributes of the device and its agents prior to use.

B. Reconstitution

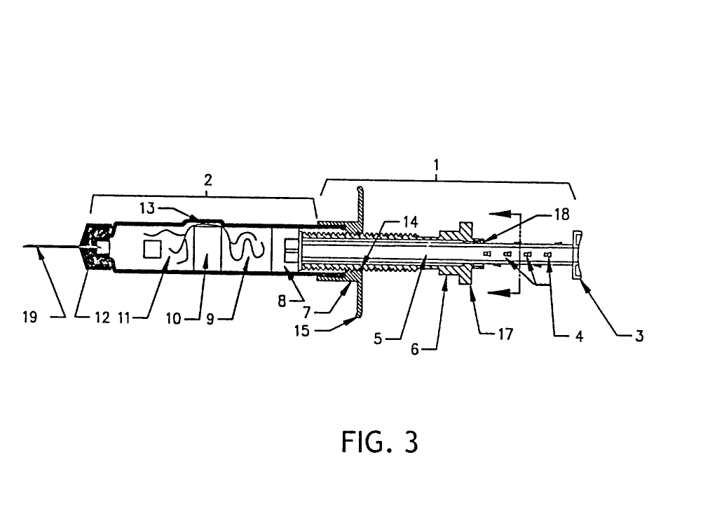

As a finished device, the syringe is ready for use after removal from its packaging, as shown in cross-section in FIG. 3. To use the syringe, a needle (19) is attached to the distal needle fitting (12) of the cartridge (2) which is designed for needle attachment as through a Luer fitting. This needle (19) is preferably a dual pointed standard gauge needle for subcutaneous injection of a pharmaceutical agent. However, one skilled in the art will readily determine the appropriate needle type depending on the intended application, including but not limited to intramuscular injection. Puncture of the distal septum (12) of the cartridge (2) allows unimpeded distal displacement of the plunger (3). Clockwise rotation of the knurled screw knob (17) of the position selector (6) results in distal movement of the plunger (3) and the two septa (8, 10) within the cartridge (FIG. 4). As the distal septum (10) reaches the region of the cartridge containing the bypass notch (18), diluent fluid flows from diluent chamber (9) into the agent-containing chamber (11) containing the dried product, beginning the process of product reconstitution. Further displacement by clockwise rotation of the position selector (6) results in the complete transfer of fluid from the diluent chamber (9) to the agent-containing chamber (11) (FIG. 5). At this point, the threads of the position selector (6) disengage from those on the base (7). Hence, additional clockwise rotation of the threaded position selector (6) does not result in further movement of the plunger (3). The dried product is formulated to completely dissolve in the diluent at this position.

While the actions of the device shown in FIGS. 3, 4 and 5 resulting in reconstitution of dried product represent a preferred embodiment of the present device, there are other applications of these functional actions that are also within the scope of the present invention. When the driver is attached to a cartridge containing aqueous liquid agent or viscous agents, the screw-driven advancement of the plunger may be used for other applications including, but not limited to, purging the cartridge of air. In such applications, it may be necessary to shorten the threaded section of the position selector (6) to limit plunger displacement that occurs through clockwise rotation of the position selector (6). Furthermore, it is also intended that the present invention be embodied by devices which lack the threaded plunger displacement mechanism entirely, containing only the stepwise displacement mechanism of sequential ejections of identical or non-identical volume from the attached cartridge. Since step displacements of the plunger may be used to reconstitute dried agent, this later embodiment is also intended to encompass devices wherein initial step-wise plunger displacements resulting from thumb pressure between the oblong plate extension and the plunger shaft can be used to reconstitute the dried agent.

C. Air Purge

Further distal displacements of the plunger beyond that achieved through screw-driven movement is accomplished through stepwise displacements utilizing the multi-dose mechanism of the present device. Such a mechanism is achieved through use of thumb pressure to advance the plunger (3) and reciprocal stops (4) that are positioned both at specific angles relative to the plunger shaft, e.g., 90°, and at specified axial, or longitudinal, positions along the plunger shaft (5) that remains externally exposed following completion of the screw-driven displacement. The distance along the plunger (3) axis separating reciprocal stops (4) determines the displacement of the plunger (3) in each step and hence the volume ejected from the attached needle (19). These volumes may be the same or different for individual steps depending on said axial positioning of reciprocal stops (4). The stops (4) are rod-like projections from the plunger shaft (5) designed to fit into a small keyway (18) within the proximal region of the threaded position selector (6) as shown in the surface views in FIGS. 1A, 1B, 3, 4, 5, and 6. Furthermore, the stopping projections (4) contain an elevation at the proximal surface such that movement to a stop position results in a snap, and locking of the plunger shaft to the position selector as the projection passes into the keyway (18) and the reciprocal stop (4) impacts the proximal surface of the threaded position selector (6) (FIG. 6). This mechanism provides unidirectional movement during the step-wise plunger displacement of the present device. Also, the audible/tactile snap at the completion of given step displacement indicates to the user that the desired action has been completed, e.g., air purge or completion of a specific product injection.

Completion of the screw-driven advancement of plunger (3) also results in alignment of dose indicating window (16) on the base (7) with the operational indicators imprinted onto the plunger shaft (5). The proper rotational alignment of the plunger (3) and the position selector (6) is indicated by, for example, “OK” when viewed through the dose-indicating window (16). The user then a) holds the syringe upward and b) depresses the plunger (3) to the next reciprocal stop (4). This results in a step displacement of the plunger (3) that expels the residual air from the cartridge and also changes the display in the dose-indicating window (16) to, for example, “AP” to indicate that the air purge has been completed and that the syringe is ready for the first injection of liquid agent. (See FIG. 6)

D. Multi-Dose Injection

Following the air purge, multi-dose injection occurs by clockwise rotation of the position selector by 90° and depression of the syringe plunger (3) to the next reciprocal stop (4) position. The clockwise rotation of the position selector (6), which occurs without like rotation of the syringe plunger (3), axially aligns the second stopping projection on the plunger shaft (5) with the keyway (18) on the position selection (6) that accommodates reciprocal stops (4). This allows displacement of the plunger (3) when thumb pressure is applied, the displacement being limited by the next reciprocal stop (4) on the plunger shaft (5) which impacts the distal surface of the position selector (6). This reciprocal stop (4) does not enter the position selector notch (18) since it is at a 90° angle to the reciprocal stop (4) now within the keyway (18) (FIG. 7). The volume of the injection is determined by the displacement of the plunger (3), which in turn is determined by the distance separating reciprocal stops (4) on the plunger shaft (5), for example, 0.126 inches as shown in FIG. 1. At the end of the first such step after the air purge, the dose-indicating (16) window will now display “1”, for example, to indicate that the first injection has been delivered and that the syringe is ready for the second injection. The second and subsequent injections are likewise initiated by clockwise rotation of the position selector (6) and application of thumb pressure to physically move the plunger (3) to the next reciprocal stop (4). The dose-indicating window (16) then shows “2” to indicate that the second injection has been delivered and that the syringe is ready for the third injection. Identical actions are followed until all injections have been delivered from the syringe.

What is claimed is:

1. A driver for delivering multiple doses of a compound from a cartridge, comprising:
a) a threaded base adapted for attachment to a cartridge;
b) a position selector comprising:
i) external threads for seating with the threads of the base;
ii) a central opening extending through the position selector;
iii) a keyway in the central opening; and
c) a plunger having a shaft, a distal end and a proximal end, wherein said plunger extends through the central opening of the position selector and is longer than the position selector, said plunger comprising projections for passage through the keyway on the position selector.

2. The multiple dose driver according to claim 1 wherein the projections are separated from each other axially and at different angles to the axis of the plunger shaft

3. The multiple dose driver according to claim 1 wherein the driver is composed of sterilizable amorphous medical plastic material.

4. A multiple dose administration device comprising the driver of claim 1 and a cartridge, wherein the cartridge comprises a single chamber.

5. The multiple dose administration device of claim 4, wherein the cartridge contains an injectable pharmaceutical formulation.

6. The multiple dose administration device of claim 4, wherein the cartridge contains any dried or liquid agent.

7. A multiple dose administration device comprising the driver of claim 1 and a dual-chamber cartridge, wherein said dual-chamber cartridge comprises a proximal chamber containing a liquid agent and a distal chamber containing a dried agent.

8. The multiple dose administration device according to claim 7 wherein said cartridge comprises a pharmaceutical ingredient.

9. The multiple dose administration device according to claim 4 or claim 8 wherein said pharmaceutical ingredient is selected from the group consisting of FSH, insulin, growth hormone, interferon, colony stimulating factor, erythropoietin, steroid hormones and an allergen.

10. A multiple dose administration device according to claim 4 or claim 7 further comprising a needle attached at a distal end of said cartridge for delivery of liquid and dried agents.

11. The multiple dose driver according to claim 1 wherein said projections are spaced equally along a longitudinal axis of said plunger shaft.

12. The multiple dose driver according to claim 1 wherein said projections are spaced along a longitudinal axis of said plunger shaft corresponding to desired dosages.

13. The multiple dose driver according to claim 1 wherein the position selector further comprises a window to allow visual access of the plunger.

14. The multiple dose driver according to claim 11 wherein the plunger shaft further comprises at least one position indicator visible through said window when said position indicator is aligned with said window.

15. The multiple dose driver according to claim 1 wherein the base further comprises first and second lateral extensions capable of supporting an index and middle finger of a human hand.

16. The multiple dose driver according to claim 1 wherein said projections comprise a proximal end and a distal end, said proximal end of said projections having a greater radial profile than said distal end of said projections.

17. The multiple dose driver according to claim 16 wherein insertion of said distal end of said projection into said keyway produces an audible or tactile stimulus.

18. The multiple dose driver according to claim 16 wherein said keyway engages said projections such that the movement of said plunger in the proximal direction is prevented.

19. A driver for delivering multiple doses of a compound from a cartridge, comprising:
a) a base adapted for attachment to a cartridge, said base comprising a central opening extending through said base and a keyway in said central opening; and
b) a plunger, wherein said plunger extends through said central opening of said base and is longer than said base, said plunger comprising projections for passage through said keyway on said base.

20. A method for ejecting a compound from a multiple-dose syringe, comprising:
a) attaching a cartridge containing the compound to the driver of claim 1;
b) rotating the position selector to align a first projection with the keyway of the position selector;
c) depressing the plunger until a second projection impacts the position selector to eject a portion of the compound;
d) rotating the position selector to align the second projection of the plunger shaft with the keyway of the position selector; and
e) depressing the plunger until a third projection impacts the position selector to eject a portion of the compound.

21. The method for ejecting a compound according to claim 20 wherein said cartridge has a first chamber containing a diluent and a second chamber containing a dried compound, further comprising, after the step of attaching a cartridge, advancing said position selector into said threaded base to reconstitute said dried compound with said diluent.

22. The method for ejecting a compound according to claim 20 further comprising, before the step of rotating the position selector to align a first projection, attaching a needle to a distal end of said cartridge for delivery of said compound.

23. The method for ejecting a compound according to claim 21 further comprising, after the step of attaching a needle, advancing said position selector into said threaded base to purge air from said cartridge.

24. The method according to claim 23 further comprising, after the step of advancing said position selector, inserting said needle into a patient.

25. The method according to claim 20 wherein said keyway engages said projections such that movement of said plunger in the proximal direction is prevented.
